(19) United States
(12) Patent Application Publication
(10) Pub. No.: US 2026/0224852 A1
(43) Pub. Date: Aug. 6, 2026

(54) ARTICULABLE TUBE ASSEMBLIES

(71) Applicant: Fortimedix Assets II B.V., Geleen (NL)

(72) Inventor: Mattheus Hendrik Louis Thissen, Swalmen (NL)

(21) Appl. No.: 19/420,036

(22) Filed: Dec. 15, 2025

Related U.S. Application Data
(60) Provisional application No. 63/904,002, filed on Oct. 23, 2025.

(30) Foreign Application Priority Data
Jan. 31, 2025 (NL) 2039715

(51) Int. Cl. A61M 25/01; A61M 25/00

(52) U.S. Cl. CPC A61M 25/0147; A61M 25/0054; A61M 2025/0063; A61M 2025/015

(57) ABSTRACT

Tubular assemblies are disclosed herein. According to some embodiments, the present technology includes an elongate member having a longitudinally extending tendon, wherein the tendon includes a tapered region along which the width of the tendon continuously decreases. The elongate member can include first and second portions on either side of the tendon and separated from the tendon by respective first and second slits. Each of the first and second slits have a slit length, the slit length being the longest distance measured across the respective slit along a line substantially parallel to the longitudinal axis of the elongate member. The tapered region can have a tapered region length greater than the slit length.

### CROSS-REFERENCE TO RELATED APPLICATION(S)

The present application claims the benefit of priority to U.S. Provisional Application No. 63/904,002, filed Oct. 23, 2025, and to Dutch Application No. 2039715, filed Jan. 31, 2025, each of which is incorporated by reference herein in its entirety.

### TECHNICAL FIELD

The present technology relates to articulable tube assemblies and associated systems and methods of use.

### BACKGROUND

Steerable catheters are widely used in minimally invasive medical procedures to navigate through tortuous anatomical pathways and reach target locations within the human body. Conventional steerable catheters typically employ steering wires or pull wires that extend along the length of the catheter shaft and are mechanically coupled to the distal tip. When tension is applied to these steering wires through a proximal control mechanism, the distal end of the catheter deflects in a predetermined direction to facilitate navigation through complex vascular or anatomical structures.

However, steering wire-based catheter systems present several technical challenges that can limit their effectiveness and precision during medical procedures. For example, the mechanical coupling between the steering wires and the catheter tip may result in unpredictable deflection characteristics. Additionally, the transmission of force from the proximal control mechanism to the distal tip through the steering wires can be inconsistent due to wire material properties and friction losses along the catheter shaft. These factors can lead to reduced responsiveness, imprecise tip positioning, and difficulty maintaining desired catheter orientations during complex navigational maneuvers, thereby potentially compromising procedural outcomes and requiring additional time and effort from medical practitioners.

Accordingly, there is a need for improved steering devices for medical procedures.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 depicts an example articulable system, in accordance with several embodiments of the present technology.

FIG. 2A depicts an exploded view of an example tube assembly, in accordance with several embodiments of the present technology.

FIG. 2B depicts a detailed view of a portion of an example tube assembly, in accordance with several embodiments of the present technology.

FIG. 3 shows an example intermediate member in accordance with several embodiments of the present technology.

FIG. 4 depicts an unwrapped view of an example tube with tendons in an example tube assembly, in accordance with several embodiments of the present technology.

FIG. 5 depicts an unwrapped view of an example tube with tendons in an example tube assembly, in accordance with several embodiments of the present technology.

FIG. 6 shows various regions of an example intermediate member for use with a tube assembly in accordance with several embodiments of the present technology.

FIG. 7A is an enlarged view of a portion of a tendon of the intermediate member of FIG. 6. In FIG. 7A, the tendon is shown in a first axial position.

FIG. 7B is an enlarged view of the portion of the tendon of FIG. 7A.

FIG. 7C is an enlarged view of the portion of the tendon of FIG. 7A, shown in a second axial position.

FIG. 7D is an enlarged view of the portion of the tendon of FIGS. 7A and 7C, shown in a third axial position.

FIG. 8 is an enlarged view of a tendon according to the prior art.

### DETAILED DESCRIPTION

As used herein, the terms “proximal” and “distal” are defined with respect to an operator, e.g., a robot or physician that operates the instrument, catheter, or endoscope. For example, a proximal portion should be construed as a portion located closer to the robot or physician and a distal portion should be construed as a portion located farther from the robot or physician.

The present technology relates to a tube for a flexible and/or articulable device such as a medical instrument or catheter (e.g., for endoscopic and/or invasive type of applications, such as in surgery) or other articulable devices. Tube assemblies according to the present technology can be used in in a variety of medical applications, including but not limited to accessing and/or navigating the vasculature (arteries and/or veins), the heart (e.g., the chambers of the heart), the respiratory system (e.g., the mouth, nose, trachea, bronchi, bronchioles, etc.) and/or the gastrointestinal system (e.g., the esophagus, small intestine, the colon, etc.). The articulable instrument can be used in both medical and non-medical applications. Examples of the latter include inspection and/or repair of mechanical and/or electronic hardware at locations that are difficult to reach (e.g., optical devices, plumbing devices, etc.). Hence, terms used in the following description such as endoscopic application or invasive instrument must be interpreted in a broad manner.

### I. Articulable Tube Assemblies

The present technology relates to a tube for an articulable device such as a medical instrument or catheter (e.g., for endoscopic and/or invasive type of applications, such as in surgery) or other articulable devices. Articulable tube assemblies according to the present technology can be used in in a variety of medical applications, including but not limited to accessing and/or navigating the vasculature (arteries and/or veins), the heart (e.g., the chambers of the heart), the respiratory system (e.g., the mouth, nose, trachea, bronchi, bronchioles, etc.) and/or the gastrointestinal system (e.g., the esophagus, small intestine, the colon, etc.). The articulable tube assemblies disclosed herein can be used in both medical and non-medical applications. Examples of the latter include inspection and/or repair of mechanical and/or electronic hardware at locations that are difficult to reach (e.g., optical devices, plumbing devices, etc.). Hence, terms used in the following description such as endoscopic application or invasive instrument must be interpreted in a broad manner.

In some embodiments, an articulable device may include an articulable tube assembly having one or more articulable regions along its length, where the one or more articulable regions are configured to articulate (e.g., bend, curve, etc.). In some embodiments, an articulable region may be configured to articulate passively (e.g., in response to articulating movement of a stylet or an outer sheath telescopically engaged with the articulable region, in response to an anatomical environment, etc.). Additionally or alternatively, in some embodiments, an articulable region may be configured to articulate in response to one or more steering inputs. For example, in some embodiments, the articulable tube assembly may be configured to receive one or more steering inputs at a first location along the length of the articulable tube assembly, and the articulable tube assembly may be configured to communicate and/or transform the steering input(s) into articulation at one or more articulable regions located at at least a second location along the length of the articulable tube assembly, where the second location is distal to the first location. In some embodiments, the one or more steering inputs may be located at a proximal portion of the articulable tube assembly, and the articulable region(s) may be located at an intermediate portion and/or distal portion of the articulable tube assembly.

In some embodiments, the articulable tube assembly may include multiple elongate members (e.g., tubular members) that are arranged coaxially, such as in a nested manner. The multiple elongate members may have respective flexible regions that are longitudinally aligned, such that when the multiple elongate members are assembled together in a tube assembly, their flexible regions are configured to be shaped and articulate in tandem. In some embodiments, an actuating input may be applied to a portion (e.g., proximal portion, intermediate portion, or other portion that is longitudinally distanced from a flexible region) of one elongate member (e.g., an outer elongate member), and that actuating input may be communicated to a steering feature such as a tendon (as described in further detail below) on an underlying elongate member to cause collective articulation of the multiple assembled elongate members. “Steerable region” is used herein to refer to both passively bendable and/or flexible regions of the articulable tube assembly and/or the elongate members, and to steerable regions configured to be deflected in a desired direction by a desired amount in response a steering input from a user. Some flexible regions may be both passively bendable and steerable, some flexible regions may be only passively bendable (i.e., not controlled by any steering tendons), and some flexible regions may bend only in response to a steering input.

Further details of example articulable devices and tube assemblies are described below. Although examples of an articulable tube assembly are primarily described herein as steerable tube assemblies, it should be understood that at least some aspects of an articulable tube assembly may be similarly incorporated in a tube assembly that is passively articulated (e.g., in response to articulating movement of a stylet arranged within the tube assembly, movement of an outer tube arranged outside the tube assembly, in response to advancement through a tortuous lumen, and/or the like).

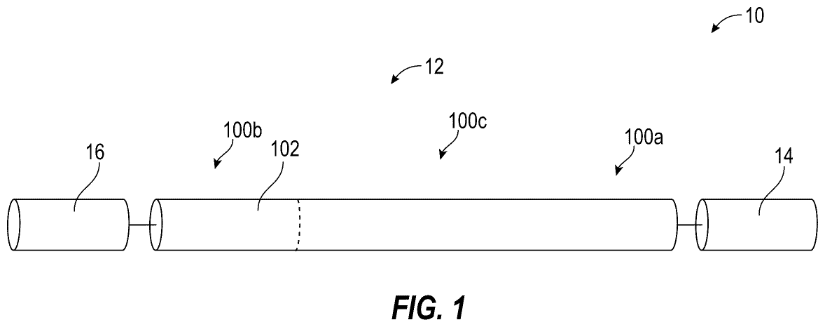

FIG. 1 is a schematic illustration of an example articulable system 10 including a steerable device 12 and at least one actuator 14 configured to be operably coupled to the steerable device 12 to provide steering input to the steerable device 12. As shown schematically in FIG. 1, the steerable device 12 may include a tube assembly 100 and at least one end effector 16 configured to be coupled to the tube assembly 100. In some embodiments, the articulable system 10 does not include the actuator 14 and/or the end effector 16.

The tube assembly 100 includes a distal portion 100b configured to be positioned in a body lumen, a proximal portion 100a configured to be coupled to a steering interface (manual or robotic), and an intermediate portion 100c extending therebetween. The tube assembly 100 includes a steerable region 102 at the distal portion 100b that is configured to be selectively deflected in response to a steering input at the proximal portion 100a, as detailed herein. The intermediate portion 100c may be passively bendable and/or flexible (e.g., to accommodate one or more turns in the body lumen) but not steerable. In some embodiments, the intermediate portion is not bendable. The steerable region 102 may be configured to deflect in a single plane (e.g., bi-directional) or multiple planes (e.g., multi-dimensional). While only a single steerable region 102 is shown in FIG. 1, in some examples the tube assembly 100 may have two or more steerable regions 102, each independently controllable at the proximal portion 100a. In some embodiments, the tube assembly 100 does not include a steerable region 102 and instead comprises a tube configured to transition between flexible and rigid configurations so as to lock the tube in a desired shape. As used herein, “medical instrument” can refer to the articulable system, the steerable device, and/or the tube assembly, whether steerable or not.

In some embodiments, the steerable device 12 and/or tube assembly 100 is configured to be coupled to the at least one actuator 14 at an end portion (e.g., proximal end portion, distal end portion) and/or at an interior and/or exterior portion of the steerable device 12 and/or tube assembly 100. Although FIG. 1 illustrates schematically an actuator 14 that is coupled end-to-end with the tube assembly 100, it should be understood that this arrangement is representative, and the actuator 14 may be coupled to the tube assembly 100 in any suitable manner. For example, the actuator 14 may be coupled to a proximal end portion of the tube assembly 100 to provide an actuating input directed radially inward toward the end portion of the tube assembly (e.g., at least a portion of the actuator 14 may be positioned radially outward of the tube assembly 100). The actuating input may include an input in a longitudinal (e.g., translational) direction and/or circumferential (e.g., rotational) direction, for example. In some embodiments, the actuating input may be manual (e.g., controlled by a human operator) and/or robotic.

As shown schematically in FIG. 1, the steerable device 12 and/or tube assembly 100 may be coupled at an end portion (e.g., a distal end portion) to the at least one end effector 16. The end effector 16 may include a tool (e.g., graspers, scissors, ablation tool, etc.), a sensor (e.g., camera, electrode, etc.) and/or any suitable instrument. Components useful for operation of the end effector 16, such as wires for actuation of a tool, signal transmission, and/or electrical power transmission, may be passed along one or more lumens defined within the tube assembly 100. In some embodiments, no end effector 16 is coupled to the steerable device 12 and/or tube assembly 100; for example, the steerable device 12 may be a catheter (e.g., delivery catheter).

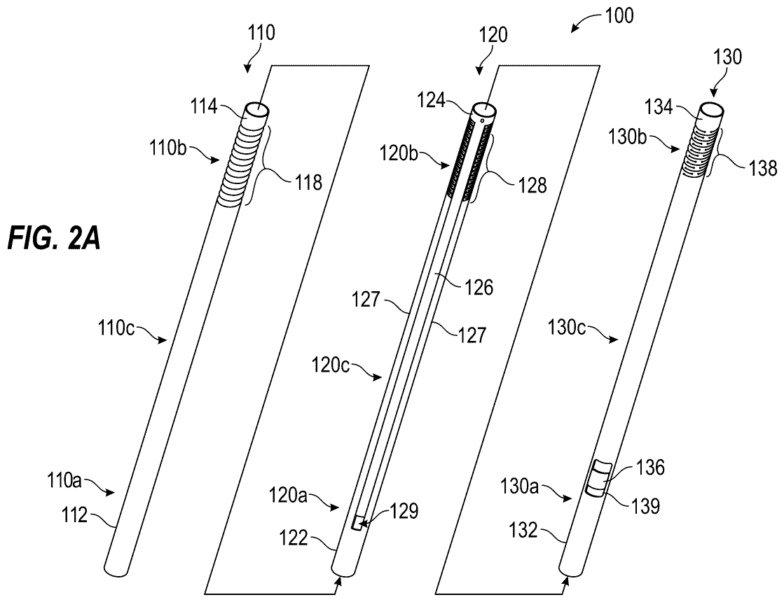

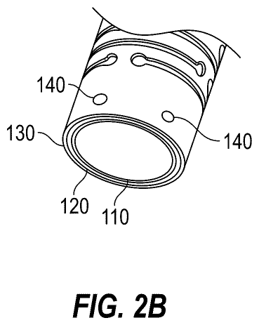

As described above, in some embodiments, a tube assembly 100 may include multiple, coaxial elongate tubular members. For example, as shown in FIG. 2A, an example tube assembly 100 may include an inner member 110, an outer member 130, and an intermediate member 120 arranged between the inner member 110 and the outer member 130. The inner member 110 may be positioned within a lumen of the intermediate member 120, and the intermediate member 120 may be positioned with a lumen of the outer member 130. Although FIG. 2A illustrates an example in which the tube assembly 100 includes a single intermediate member 120, it should be understood that in some embodiments, the tube assembly 100 may include two, three, or more intermediate members arranged between the inner member 110 and the outer member 130, where each intermediate member may have a respective set of operable features. Moreover, a tube assembly 100 may include any suitable number of nested, coaxial tubular members (e.g., two, three, four, or more than four); however, for the sake of simplicity of explanation, a representative tube assembly 100 with three elongate members is primarily described herein. In some embodiments, an articulable device may include only a single tubular member incorporating at least some features of the elongate members as described herein.

The inner member 110 may include a proximal portion 110a including a proximal end portion 112, a distal portion 110b including a distal end portion 124, and an intermediate portion 110c extending between the proximal and distal portions 110a, 110b of the inner member 110. The proximal portion 110a may be aligned with the proximal portion 100a of the tube assembly 100, the intermediate portion 110c may be aligned with the intermediate portion 100c of the tube assembly 100, and the distal portion 110b may be aligned with the distal portion 100b of the tube assembly 100. The distal portion 110b of the inner member 110 may include at least one flexible region 118 configured to articulate (e.g., bend, curve, flex, etc.) and that is axially aligned with the steerable region 102 when the tube assembly 100 is assembled. The distal end portion 114 of the inner member 110, which is distal of the flexible region 118, may comprise a rigid (e.g., non-articulable) band. Although FIG. 2A illustrates an example in which the inner member 110 includes one flexible region 118 located generally in the distal portion 110b, it should be understood that in some embodiments, the inner member 110 may include two or more (e.g., two, three, four, more than four, etc.) flexible regions 118 arranged along the distal portion 110b. In some embodiments, adjacent flexible regions 118 can be separated by a rigid portion and/or band.

The intermediate portion 110c of the inner member 110 may be passively bendable and/or flexible (e.g., to accommodate one or more turns in the body lumen) but not steerable. In some embodiments, the intermediate portion 110c may be rigid and thus not bendable (e.g., maintains a linear configuration).

The flexible region 118 may include one or more articulating features configured to enable the flexible region 118 to assume a suitable articulated shape (e.g., bent, curved, etc.). For example, as shown in FIG. 2A, the flexible region 118 may include a plurality of cuts and/or slits, such as circumferentially extending cuts and/or helically extending cuts, etc., configured to improve the flexibility and/or bendability of the respective elongate member along that region. As described in further detail herein, the cuts may, for example, be formed by cutting any suitable pattern from the wall of the inner member 110. In some embodiments, dimensional aspects of the cut pattern in the flexible region 118 (e.g., longitudinal length of the flexible region 118, size and shape of cuts, spacing between cuts, etc.) may be selected to accommodate specific performance requirements, such as with regard to bending angle, bending flexibility, longitudinal stiffness, and/or radial stiffness of the flexible region 118.

The intermediate portion 110c of the inner member 110 may also include one or more articulating features, similar to the articulating features described above, that enables the intermediate portion 110c to passively bend and flex.

Similar to the inner member 110, the outer member 130 may include a proximal portion 130a including a proximal end portion 132, a distal portion 130b including a distal end portion 134, and an intermediate portion 130c extending between the proximal and distal portions 130a, 130b of the outer member 130. The proximal portion 130a may be aligned with the proximal portion 100a of the tube assembly 100, the intermediate portion 130c may be aligned with the intermediate portion 100c of the tube assembly 100, and the distal portion 130b may be aligned with the distal portion 100b of the tube assembly 100. The distal portion 130b of the outer member 130 may include at least one flexible region 138 configured to articulate (e.g., bend, curve, flex, etc.) and that is axially aligned with the steerable region 102 when the tube assembly 100 is assembled. The distal end portion 134 of the outer member 130, which is distal of the flexible region 138, may comprise a rigid (e.g., non-articulable) band. Although FIG. 2A illustrates an example in which the outer member 130 includes one flexible region 138 located generally in the distal portion 130b, it should be understood that in some embodiments, the outer member 130 may include two or more (e.g., two, three, four, more than four, etc.) flexible regions 138 arranged along the distal portion 130b. In some embodiments, adjacent flexible regions 138 can be separated by a rigid portion and/or band.

The one or more flexible regions 138 of the outer member 130 may be constructed in a similar manner to the flexible region(s) 118 of the inner member 110. The flexible regions 118 and 138 may have the same or different cut configurations and/or cut patterns.

The intermediate portion 130c of the outer member 130 may be passively bendable and/or flexible (e.g., to accommodate one or more turns in the body lumen) but not steerable. The intermediate portion 130c may also include one or more articulating features, similar to the articulating features described above, that enables the intermediate portion 130c to passively bend and flex. In some embodiments, the intermediate portion 130c may be rigid and thus not bendable (e.g., maintains a linear configuration).

The intermediate member 120 may include a proximal portion 120a including a proximal end portion 122, a distal portion 120b including a distal end portion 124, and an intermediate portion 120c extending between the proximal and distal portions 120a, 120b of the intermediate member 120. The proximal portion 120a may be aligned with the proximal portion 100a of the tube assembly 100, the intermediate portion 120c may be aligned with the intermediate portion 100c of the tube assembly 100, and the distal portion 120b may be aligned with the distal portion 100b of the tube assembly 100. The distal portion 120b of the intermediate member 120 may include at least one flexible region 128 configured to articulate (e.g., bend, curve, flex, etc.) and that is axially aligned with the steerable region 102 when the tube assembly 100 is assembled. The distal end portion 124 of the intermediate member 120, which is distal of the flexible region 128, may comprise a rigid (e.g., non-articulable) band. Although FIG. 2A illustrates an example in which the intermediate member 120 includes one flexible region 128 located generally in the distal portion 120b, it should be understood that in some embodiments, the intermediate member 120 may include two or more (e.g., two, three, four, more than four, etc.) flexible regions 128 arranged along the distal portion 120b. In some embodiments, adjacent flexible regions 128 can be separated by a rigid portion and/or band.

The one or more flexible regions 128 of the intermediate member 120 may be constructed in a similar manner to the flexible region(s) 118 of the inner member 110. The flexible regions 118, 128, and 138 may have the same or different cut configurations and/or cut patterns.

The intermediate portion 120c of the intermediate member 120 may be passively bendable and/or flexible (e.g., to accommodate one or more turns in the body lumen) but not steerable. The intermediate portion 120c may also include one or more articulating features, similar to the articulating features described above, that enables the intermediate portion 120c to passively bend and flex. In some embodiments, the intermediate portion 120c may be rigid and thus not bendable (e.g., maintains a linear configuration).

The inner member 110, the intermediate member 120, and the outer member 130 may be assembled to form a combined unit that defines the tube assembly 100. For example, the inner member 110 may be inserted into the intermediate member 120, and the combined inner member 110 and intermediate member 120 subassembly may be inserted into the outer member 130, although any order of insertion may be possible. In some embodiments, only two elongate members (e.g., the inner member 110 and the intermediate member 120, the inner member 110 and the outer member 130, or the intermediate member 120 and the outer member 130) may be assembled to form a combined unit within the steerable tubular assembly. In some embodiments, the distal end portions 114, 124, 134 of the inner member 110, the intermediate member 120, and/or the outer member 130, respectively, may be coupled to one another so as to be fixed together. In some examples, the proximal end portions 112, 122, 132 of the inner member 110, the intermediate member 120, and/or the outer member 130, respectively, may be coupled to one another so as to be fixed together. Moreover, the inner member 110, intermediate member 120, and outer member 130 may be fixed to one another at other locations along their respective lengths. For example, in those embodiments in which the tube assembly 100 includes two steerable regions (and thus each of the inner member 110, intermediate member 120, and outer member 130 have two flexible regions, each of which is longitudinally aligned with the corresponding flexible region in the radially adjacent tube), the inner member 110, the intermediate member 120, and the outer member 130 may be fixed to one another at the respective rigid bands on either side of each flexible region.

Such coupling of the inner member 110, the intermediate member 120, and/or the outer member 130 may be accomplished in any suitable manner, such as with epoxy, welding (e.g., laser welding), or mechanical interfit (e.g., press fit). For example, FIG. 2B provides a more detailed view of a distal end portion of an example tube assembly including three co-axially arranged layers or tubular members, including inner member 110, intermediate member 120, and outer member 130. The distal end portions 114, 124, 134 of the inner member 110, the intermediate member 120, and the outer member 130 may be fixedly attached to one another, such as with one or more spot welds 140. In some embodiments, multiple spot welds 140 may be arranged circumferentially around the tube assembly, either equally distributed or unequally distributed around the circumference of the tube assembly 100. The spot welds 140 may be arranged at the proximal end portions 112, 122, and/or 132, and/or at the distal end portions 114, 124, and/or 134, and/or at any suitable axial location along the length of the tube assembly 100.

As shown in FIG. 2A, the intermediate member 120 may further include one or more tendons 126, each associated with and/or extending through a respective flexible region 128, such that movement of a tendon 126 causes articulation and/or deflection of its corresponding flexible region 128. In some embodiments, a distal end of a tendon 126 may be coupled to (e.g., integrally formed with, or attached such as via at least one weld) a rigid portion and/or band just distal of the corresponding flexible region 128. For example, in FIG. 2A, the distal end of tendon 126 is integral with the rigid band comprising the distal end portion 124 of the intermediate member 120.

A tendon 126 may be formed as a longitudinal member or strip extending longitudinally along at least a portion of a wall of the intermediate member 120, for at least a portion of the length of the intermediate member 120. A tendon 126 may be cut from the sidewall of the intermediate member 120 and thus integral with the sidewall of the intermediate member 120. A tendon 126 may be configured to move generally in a longitudinal direction within a respective slot 129 defined by the intermediate member 120 (e.g., cut from the wall of the intermediate member 120). On either side of a tendon 126 or slot 129 can be a tendon-adjacent portion 127 of the sidewall that is not configured to move longitudinally and/or be actuated and is separated from the tendon 126 by a slit. The slit may, for example, be formed by removal of material such as laser cutting, where the width of the slit corresponds to the width of the laser beam. In the example shown in FIG. 2A, the slot 129 is generally linear and extends in a longitudinal direction. However, in some embodiments the slot 129 may be any suitable shape generally extending in a longitudinal direction, such as a helical shape that wraps in a spiral manner around the wall of the intermediate member 120.

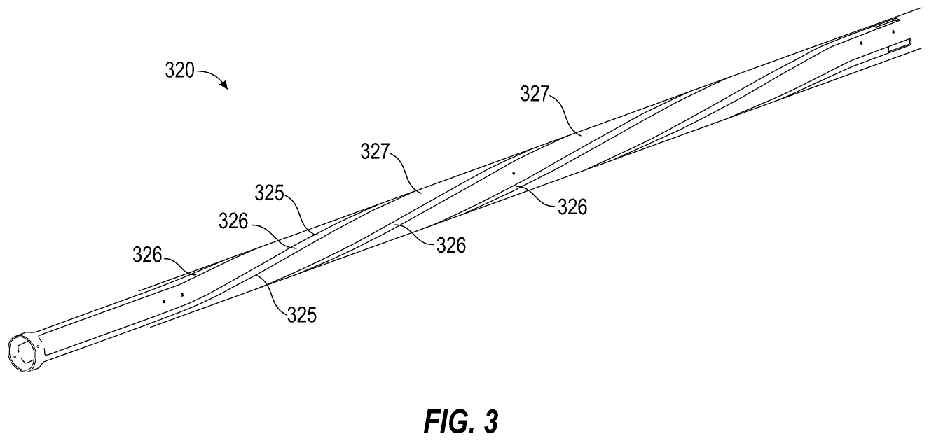

A tendon 126 may have any suitable corresponding shape to travel within the corresponding slot 129 (e.g., a helical tendon configured to travel generally in a proximal and/or distal direction within a helical slot). For example, FIG. 3 illustrates an example intermediate member 320 (an example of intermediate member 120) that includes multiple helical tendons 326 that have been obtained after making longitudinal cuts 325 (only a few labeled) in the wall of the intermediate member 320. As shown in FIG. 3, the tendons 326 are, at least in part, spiraling about a longitudinal axis of the intermediate member 320 such that a proximal portion of a given tendon 326 is arranged at a different angular orientation about the longitudinal axis than a distal portion of the same given tendon 326. The tendon-adjacent portions 327 of the sidewall may also extend helically and/or spiraling. In some embodiments, the spiral construction may be such that the proximal end portion of a tendon 326 is arranged at an angularly shifted orientation of 180 degrees about the longitudinal axis relative to the distal end portion of the same tendon 326. However, the angularly shifted orientation between the two end portions of a tendon 326 may be any suitable angle (e.g., 45 degrees, 60 degrees, 75 degrees, 90 degrees, etc.). The spiraling compensates path length changes in the tendons 326 caused by any bending of the intermediate portion of the intermediate member 320, for instance when the tube assembly is inserted in a tortuous path.

In some embodiments, a tendon 126 may be actuated via an actuating input applied to a feature of the outer member 130. For example, as shown in FIG. 2A, in some embodiments, the outer member 130 may include at least one slider 136 configured to move within a respective slot 139 defined by the outer member 130. Each slider 136 may be coupled to an underlying tendon 126 (e.g., via epoxy, spot welding, etc.) such that movement of the slider 136 results in movement of its associated tendon 126. The outer member 130 may include multiple sliders 136, where each slider 136 is coupled to a respective tendon 126, such as selective actuation of the sliders 136 results in selective movement of tendons 126 and thus selective articulation of the articulable regions of the members of the tube assembly. Although the slot 139 is shown in FIG. 2A as generally linear such that the slider 136 moves longitudinally along the outer member 130 (e.g., to push and/or pull a tendon 126 to which the slider 136 is coupled), it should be understood that in some embodiments, the slider 136 may move at least partially in a rotational manner in an arcuate slot 139 (e.g., the slider 136 may be configured to move in a helical manner along a helical slot 139, which may, for example, thereby cause movement of an underlying tendon 126 to which the slider 136 is connected).

Furthermore, although only one tendon 126 is visible in the example shown in FIG. 2A, the intermediate member 120 may include any suitable number of tendons 126. For example, in some embodiments a second tendon 126 may be located on a circumferentially opposite side of the intermediate member 120. For example, two tendons 126 may be circumferentially offset about 180 degrees from one another and operate in an antagonistic manner to articulate an articulable region 128 to which the two tendons 126 are coupled. For instance, a first tendon 126 may be pulled proximally in a longitudinal direction (and/or a second tendon 126 arranged circumferentially opposite to the first tendon 126 may be pushed distally in a longitudinal direction) to cause bending of the articulable region 128 in a first direction. The second tendon 126 may be pulled proximally in a longitudinal direction (and/or the first tendon 126 may be pushed distally in a longitudinal direction) to cause bending of the articulable region 128 in a second direction (e.g., opposite the first direction).

In some embodiments, the intermediate member 120 may include three or more tendons 126 coupled to a common articulable region 128. In some embodiments, multiple tendons 126 may be circumferentially spaced apart in an equidistant manner, though in some embodiments an intermediate member 120 may additionally or alternatively include multiple tendons 126 that are circumferentially spaced apart in an unequal manner.

Additionally or alternatively, the intermediate member 120 may include one or more tendons 126 each coupled to a different flexible region 128. For example, a first pair of antagonistic tendons 126 may be coupled to a first flexible region 128 (e.g., arranged at a first axial location along the length of the intermediate member 120), and a second pair of antagonistic tendons 126 may be coupled to a second flexible region 128 (e.g., arranged, at a second axial location along the length of the intermediate member 120). In some embodiments, the intermediate member 120 may include multiple tendons 126 of substantially equal length, though in some embodiments the intermediate member 120 may additionally or alternatively include multiple tendons 126 of different lengths.

In some embodiments, flexible region(s) of the inner member 110, the intermediate member 120, and/or the outer member 130 may be longitudinally aligned with or at least overlap with one another when the inner, intermediate, and outer members are assembled together in the tube assembly 100. As such, when the flexible region(s) 128 are articulated (via actuation of one or more tendons 126), the underlying flexible region(s) 118 of the inner member 110 and the overlying flexible region(s) 128 of the outer member 130 are passively articulated to follow or generally match the shape of the flexible region(s) 128 of the intermediate member 120.

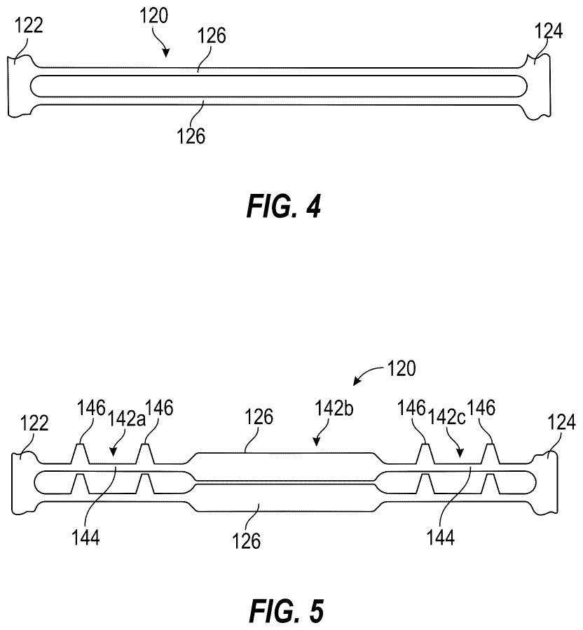

In some embodiments, the intermediate member 120 may include one or more tendons 126 having a substantially uniform cross-section (e.g., width) along its length. For example, FIG. 4 illustrates an example of an intermediate member 120 in an unrolled condition including two parallel tendons 126 having substantially uniform cross-sections along their lengths between the proximal end portion 122 of the intermediate member and the distal end portion 124 of the intermediate member. In the example shown in FIG. 4, the tendons 126 are attached at both the proximal end portion 122 and the distal end portion 124; however, in some embodiments such as that shown in FIG. 2A, the tendons 126 may be attached to only one of the proximal end portion 122 and the distal end portion 124 (e.g., only the distal end portion 124, such as to allow for articulation of an articulable region 128 near the distal end portion 124). The tendons 126 are shown as equally spaced apart, though as described above, in some embodiments, the tendons 126 may be unequally spaced apart. Additionally or alternatively, although the intermediate member 120 shown in FIG. 4 includes two tendons 126, in some embodiments the intermediate member 120 may include three, four, five, six, seven, eight, or more than eight tendons 126.

In some embodiments, one or more tendons 126 may have a varying cross-section (e.g., width) along their lengths. For example, a tendon 126 may have a wider width (e.g., as measured circumferentially in arc length around the intermediate member) at one longitudinal location, compared to its width at another longitudinal location. A wider portion of the tendon 126 may, in some embodiments, function as a spacer between adjacent tendons 126, to help prevent adjacent tendons 126 from buckling in a tangential (e.g., circumferential) direction (e.g., when pushed). However, the intermediate member 120 may include one or more spacers that are implemented in any suitable manner.

For example, FIG. 5 illustrates an example portion of an intermediate member 120 including two adjacent tendons 126 in an unrolled condition. Each tendon 126 includes a first segment 142a, a second segment 142b, and a third segment 142c that are arranged end-to-end along the tendon 126. In the second segment 142b, adjacent tendons 126 are nearly touching each other in the tangential direction such that only a narrow slot is present between the adjacent tendons 126 with a width just sufficient to allow independent movement of each tendon 126. The narrow slot may, for example, be formed by removal of material such as laser cutting, where the width of the slot corresponds to the width of the laser beam.

In the first segment 142a and the third segment 142c, each tendon 126 includes a flexible portion 144 and one or more spacers 146. The flexible portion 144 has a narrower width than the second segment 142b and narrower width than the spacer(s) 146, such that there is a wider gap between adjacent tendons 126. The one or more spacers 146 extend in a tangential direction and may be almost completely bridging the gap between adjacent flexible portions 144 in adjacent tendons 126. The spacer(s) 146 may function to suppress the tendency of the tendons 126 to shift in a tangential direction, thus improving control of the tendons 126 in a tangential direction during actuation of the tendons 126, thereby leading to more control in articulating the articulable region(s) of the tube assembly. The exact shape of the spacer(s) 146 may vary. For example, as shown in FIG. 5 the spacers 146 may have a generally triangular shape, but may alternatively have any suitable shape (e.g., rectangular, semi-circular, etc.). In some embodiments, the spacers 146 may be integrally formed with one or more tendons 126. In some embodiments, the spacers comprise the tendon-adjacent portions of the sidewall.

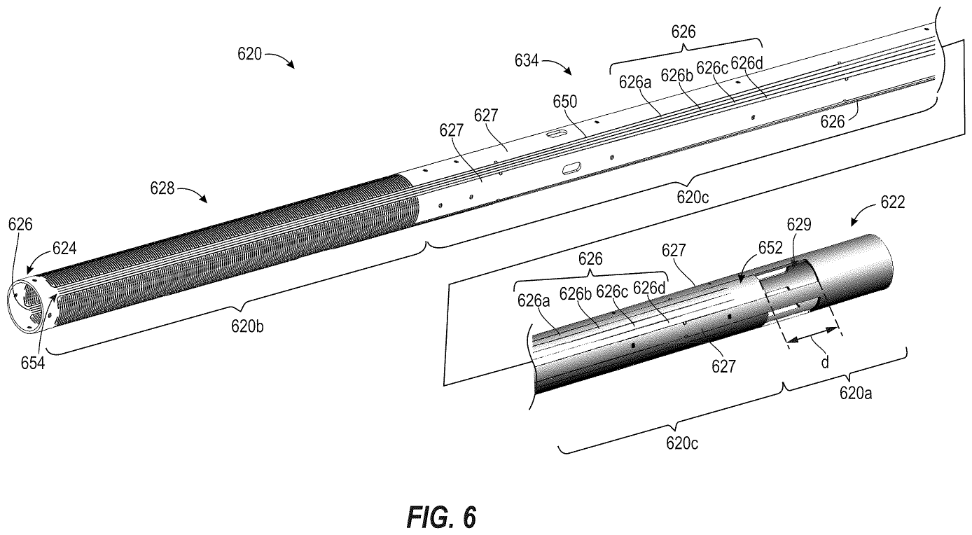

The intermediate member 120 may include any suitable spacers and/or other features, such as those described in FIGS. 4-16 of International Patent Application Publication No. WO2009/112060, FIGS. 6-10B of International Patent Application Publication No. WO2017/082720, FIGS. 15-17 of International Patent Application Publication No. WO2018/067004, FIGS. 5A-10 of International Patent Application Publication No. WO201/9009710, FIGS. 11E-14B, 17B, and 19A-20D of International Patent Application Publication No. WO2020080938, FIG. 9B of International Patent Application Publication No. WO2020/214027, FIGS. 10-19 of International Patent Application Publication No. WO2023/113598, or several figures in International Patent Application Publication No. WO2023/287289 and International Patent Application Publication No. WO2025026670, each of which is incorporated in its entirety herein by this reference.

In some embodiments, the inner member 110, the intermediate member 120, and/or the outer member 130 may include one or more tendons configured to directly engage a portion of the same elongate member and/or a radially adjacent elongate member to lock the tube assembly in a particular geometry and/or bend angle. Additional details of locking tendons are described herein.

The inner member 110, intermediate member 120, and/or outer member 130 may be formed from any suitable rigid material such as stainless steel, cobalt-chromium, shape memory alloy such as Nitinol®, plastic, polymer, composites and/or other materials. Additionally or alternatively, the elongate member(s) (e.g., inner member 110, intermediate member 120, and/or outer member 130) can be made by a 3D printing process or other known material deposition processes.

In some embodiments, one, some, or all of the elongate members comprising the tube assembly may have a wall thickness (e.g., measured in a radial direction) of about 0.03 mm to about 2.0 mm, about 0.03 mm to about 1.0 mm, about 0.05 mm to about 0.5 mm, or 0.08 mm to about 0.4 mm. Additionally or alternatively, one, some, or all of the elongate members comprising the tube assembly may have a diameter of about 0.5 mm to about 20 mm, about 0.5 mm to about 10 mm, or about 0.5 mm to about 6 mm. Additionally or alternatively, adjacent coaxial tubes may have a radial play of about 0.01 mm to about 0.3 mm.

In some embodiments, various features of the elongate members (e.g., inner member 110, intermediate member 120, and/or outer member 130) of the tube assembly 100 (e.g., articulable regions, tendons, sliders, slots, spacers, etc.) may be formed through removal of material from the wall of each respective tube forming the members of the tube assembly 100. For example, starting from a cylindrical tube with desired inner and outer diameters (and desired wall thickness), various features of an elongate member (e.g., inner member 110, intermediate member 120, outer member 130) may be formed by removing parts of the wall of the cylindrical tube, such as by laser cutting or water cutting. However, in some embodiments the elongate members may be formed through injection molding, plating techniques, 3D printing or other material deposition process, photochemical etching, deep pressing, conventional chipping techniques such as drilling or milling, and/or any suitable technique. In some embodiments, removal of material may be performed through laser cutting, which may allow for a very accurate and clean removal of material under reasonable economic conditions. In some embodiments, forming each elongate member (e.g., inner member, intermediate member, outer member) of the articulable tube assembly from a respective tube (e.g., metal tube, such as stainless steel) may enable each separate elongate member to maintain a relatively stable tubular form (e.g., flexible, but manipulable similar to a solid tube). With the elongate members being easily handled, they can be more easily assembled (e.g., nested within each other in a telescopic or concentric manner) into the articulable tube assembly.

The above-mentioned processes may be convenient ways to form each of the inner member 110, the intermediate member 120, and/or the outer member 130 in one overall process, without requiring additional steps for connecting different features of each elongate member. This simplified manufacturing process is advantageously contrasted from multiple steps that are required in manufacturing conventional steerable instruments such as with conventional steering cables, as steering cables must be connected in some way at end regions of a steerable catheter.

The inner and/or outer diameters of the members 110, 120, 130 may be selected such that at any given location along the assembled tube assembly 100, the outer diameter of the inner member 110 is slightly less than the inner diameter of the intermediate member 120, the outer diameter of the intermediate member 120 is slightly less than the inner diameter of the outer member 130, in such a way that a sliding movement of the adjacent members with respect to each other is possible. The dimensioning should be such that a sliding fit is provided between adjacent elongate members. A clearance between adjacent elongate members may generally be in the order of 0.02 to 0.1 mm, but may depend on the specific application and material used. The clearance may be smaller than a wall thickness of the tendons 126 to prevent an overlapping configuration thereof. Restricting the clearance to about 30% to 40% of the wall thickness of the tendons 126 may, for example, be generally sufficient.

Specific dimensions and features of the members 110, 120, and 130 (and/or other elongate members) may vary depending on the application in which the tube assembly 100 may be used. For example, the tube assembly may have a longer flexible portion which may help facilitate the use of the tube assembly in areas of a human body that are navigable in tortuous spaces (e.g., colon, esophagus, curved blood vessels, etc.).

According to some examples of the present technology, one or more of the elongate members of a tube assembly may include one or more tendons having a width that tapers along all or a portion of the length of the tendon and/or along all or a portion of the length of the corresponding elongate member. FIG. 6, for example, shows an example intermediate member 620 for use with any of the tube assemblies of the present technology, including those described above. The features of the intermediate member 620 can be generally similar to the features of the intermediate members described above with reference to FIGS. 1-5. Accordingly, like numbers (e.g., a flexible region 128 versus a flexible region 628) are used to identify similar or identical components in FIGS. 1-8.

The intermediate member 620 can have a proximal portion 620a, a distal portion 620b, and an intermediate portion 620c extending between the proximal and distal portions 620a, 620b. Only proximal and distal regions of the intermediate portion 620c are shown in FIG. 6 for ease of illustrating both ends of the intermediate member 620 on a single page.

The intermediate member 620 includes a flexible region 628 at the distal portion 620b, a rigid distal end portion 624, a rigid proximal end portion 622, a plurality of tendons 626 disposed in corresponding slots 629, and a plurality of tendon-adjacent regions 627 separated from the tendons 626 by slits 650. Each of the tendons 626 has a proximal end portion 652 at or near the proximal portion 620a of the intermediate member 620 and a distal end portion 654 at the distal portion 620b of the intermediate member 630. A distal portion of each of the tendons 626 passes through the flexible region 628. Each of the distal end portions 654 of the tendons 626 are fixed to and/or integral with a rigid band at the distal end portion 624 while the proximal end portions 652 are free to slide within their corresponding slots 629. As manufactured and/or in a straight (e.g., unbent and/or neutral) configuration (as shown), the proximal end portions 652 of the tendons 626 are separated from the proximal end of their corresponding slots 629 by a distance d.

While FIG. 6 shows only a single flexible region 620b, the intermediate member 620 and/or associated tube assembly can include a plurality of flexible regions (e.g., two flexible regions, three flexible regions, four flexible regions, etc.). Axially adjacent flexible regions may be separated by a rigid band, and in some embodiments the intermediate member 620 is fixed to an adjacent elongate member (e.g., an inner member, another intermediate member, and/or an outer member) at some or all of the rigid bands (e.g., via welding or other techniques, as discussed herein).

Referring still to FIG. 6, in some examples, each of the tendons 626 may be split into multiple branches 626a-626d between their respective proximal and distal end portions 652, 654. While each of the tendons 626 shown in FIG. 6 is split into four branches 626a-626d, in other embodiments the tendons 626 may have more or fewer branches (e.g., two branches, three branches, five branches, six branches, etc.). Moreover, in some implementations, at least one of the tendons 626 is split (e.g., into any number of branches) while at least another one of the tendons 626 is not split (e.g., solid along its length). In any of the foregoing embodiments, the branches may terminate at any location along the length of the intermediate member 620, including along the flexible region 628, along the distal portion 620b, along the intermediate portion 620c, or along the proximal portion 620a. In yet other examples, none of the tendons 626 have a split configuration.

In those embodiments in which one, some, or all of the tendons 626 are split into branches, the branched tendons may be coupled to a rope equalizer, for example as detailed in U.S. Patent Publication No. 2025/0176801, filed Jan. 31, 2025, which is incorporated by reference herein in its entirety.

Whether the tendons 626 are branched or not, axial movement of a tendon 626 causes articulation and/or deflection of its corresponding flexible region 628, as well as the flexible regions of any radially adjacent tube and the steerable region of the associated tube assembly. Axial movement of the tendons 626 can be controlled at the proximal portion of the tube assembly. As discussed elsewhere herein, the proximal portions of the tendons 626 can be directly or indirectly (e.g., via a portion of a radially adjacent elongate tubular member, such as an outer member, an inner member, or another intermediate member) coupled to a manually and/or robotically controlled steering interface. A predetermined longitudinal displacement of a tendon 626 will cause a predetermined amount of deflection at the corresponding steerable region. The maximum deflection angle of the steerable region depends on the design of the flexible regions of the elongate members of the tube assembly. Parameters include the number of coaxial tubes at the location of the steerable region and the type of cuts in the coaxial tubes at the flexible regions. The maximum deflection angle in turn determines the maximum longitudinal displacement of the tendons 626 associated with a particular steerable region.

As previously discussed, the tendons 626 can be arranged in pairs. Within each pair, the tendons 626 may be circumferentially offset about 180 degrees from one another to operate antagonistically to articulate the flexible region 628. In some implementations, the intermediate member 620 may have more or fewer tendons (e.g., two tendons, three tendons, five tendons, six tendons, eight tendons, etc.). Moreover, while the intermediate member 620 shown in FIG. 6 includes a single flexible region 628, in other embodiments the intermediate member 620 may have multiple flexible regions 628 (e.g., two articulable regions, three articulable regions, four articulable regions, etc.). Each flexible region 628 can be associated with two or more tendons 626, where each of the two or more tendons 626 extends through the flexible region 628 and terminates distally at a distal end of the flexible region 628. The intermediate member 620 shown in FIG. 6 includes four tendons 626 arranged approximately every 90 degrees around the circumference of the intermediate member 620. As such, the flexible region 628 and/or steerable region is configured to deflect in multiple planes (e.g., multi-dimensional). In some embodiments, the tendons 626 may have any suitable spacing, which may be uniform or not. Likewise, in some examples the flexible region 628 has only two tendons and/or is configured to deflect in a single plane.

Although not shown in FIG. 6, for those embodiments in which the intermediate portion of the tube assembly is bendable, the intermediate member 620 may additionally or alternatively include one or more locking tendons configured to be actuated at the proximal portion of the tube assembly to selectively rigidize the intermediate member 620 in a desired (bent or unbent) shape. While distal end portions 654 of the tendons 626 are operatively coupled to the flexible region(s) 628 at the distal portion 620b of the intermediate member 620, the locking tendons have distal end portions 654 that are not operatively coupled to the distal portion 620b of the intermediate member 620 and/or are not associated with causing deflection of the steerable region 602. Each locking tendon may be formed as a longitudinal member or strip extending longitudinally along at least a portion of a wall of the intermediate member 620, for at least a portion of the length of the intermediate member 620. A locking tendon may be cut from the sidewall of the intermediate member 620 and thus integral with the sidewall of the intermediate member 620. A locking tendon may be configured to move generally in a longitudinal direction within a respective slot defined by the intermediate member 620 (e.g., cut from the wall of the intermediate member 620). On either side of a locking tendon or associated slot can be a tendon-adjacent portion of the sidewall that is not configured to move longitudinally and/or be actuated and is separated from the locking tendon by a slit. The slit may, for example, be formed by removal of material such as laser cutting, where the width of the slit corresponds to the width of the laser beam. The locking tendon and associated slots and slits may be linear or may be any suitable shape generally extending in a longitudinal direction, such as a helical shape that wraps in a spiral manner around the wall of the intermediate member 620.

Similar to the steering tendons 626, a proximal end portion of a locking tendon may be disposed at the proximal portion 620a of the intermediate member 620 and/or proximal portion of the tube assembly and is manipulatable at the proximal portion by a user and/or robot (directly or through a steering interface) to move the locking tendon axially. The distal end portions of the locking tendons are disposed along the flexible intermediate portion 620c of the intermediate member 620 and/or intermediate portion of the tube assembly. Axial movement of a locking tendon in a first direction (distal or proximal) may cause the intermediate portion 620c and/or intermediate portion of the tube assembly to lock in the shape assumed by the intermediate portion 620c and/or intermediate portion of the tube assembly at the time of actuating the locking tendon. Axial movement of the locking tendon in a second direction opposite the first direction may cause the intermediate portion 620c and/or intermediate portion of the tube assembly to unlock and assume a flexible configuration once again. Additional details can be found in PCT Publication No. WO 2023287289, filed Jul. 14, 2022, as well as U.S. Provisional Application No. 63/904,058, filed Oct. 23, 2025, both of which are incorporated by reference herein in their entireties.

For the locking tendons to cause the intermediate portion 620c and/or intermediate portion of the tube assembly to rigidize or lock in a given shape, the locking tendons must travel a predetermined axial distance. The same predetermined axial distance may be required for unlocking the intermediate portion 620c and/or intermediate portion of the tube assembly. As used herein, “locking distance” refers to the predetermined axial displacement of a locking tendon that is required to lock or unlock the intermediate portion 620c and/or intermediate portion of the tube assembly. Also as used herein, “deflection distance” refers to the predetermined axial displacement of a steering tendon, measured from an as-manufactured and/or neutral state, that is required to cause maximum deflection of the corresponding steerable region of the tube assembly. “Functional distance,” as used herein, refers to the locking distance, the steering distance, and/or an axial displacement required of the tendon to perform a predetermined function as intended during operation of the tube assembly. In general, a functional distance required of any type of tendon may be about 1 mm to about 20 mm.

While the discussion below of tapered tendons is had with respect to a steering tendon 626, it will be appreciated that the various features are equally applicable to a locking tendon. Moreover, the tendon(s) of the present disclosure may have any other desired function, e.g., steering a tool arranged at the distal end of the instrument.

As shown in FIG. 6, a width of one, some, or all of the tendons 626 (and associated tendon branches 626a-626d) may taper in a longitudinal direction. The taper may extend in a distal direction (i.e., the distal end of the tapered length has a tangential width that is less than the proximal end of the tapered length) or may extend in a proximal direction (i.e., the proximal end of the tapered length has a tangential width that is less than the distal end of the tapered length). In some cases, one or more of the tendons 626 can have one or more distally tapering portions and one or more proximally tapering portions.

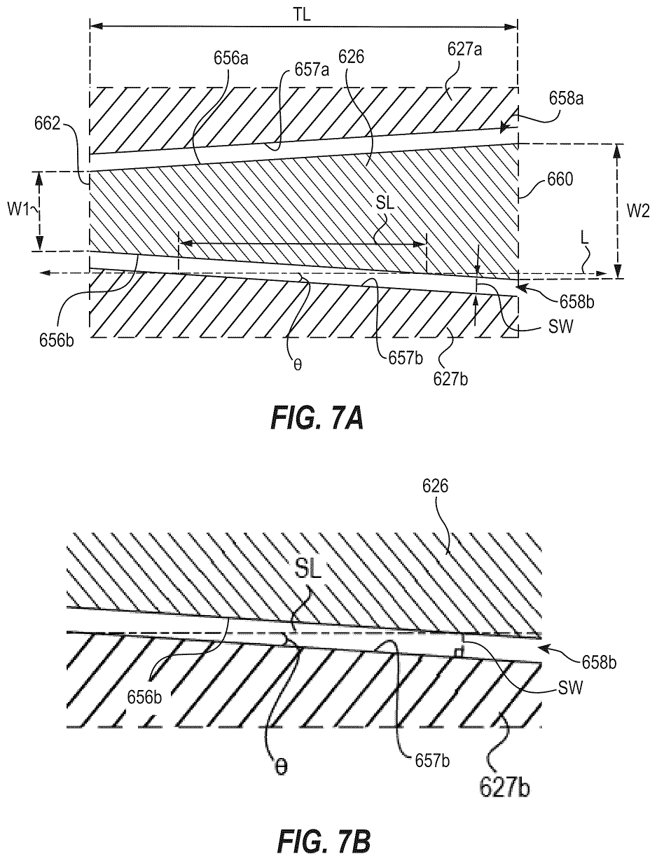

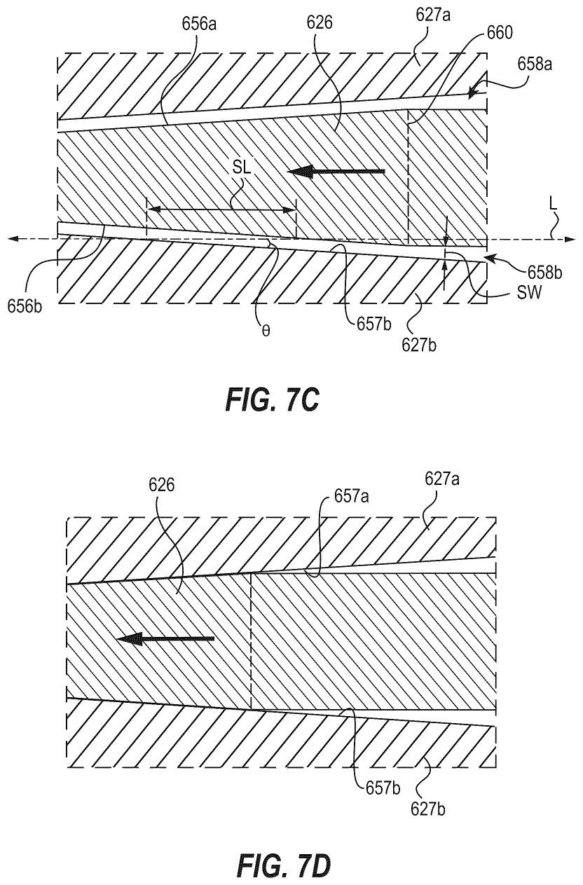

The tapered region may extend the entire length of the tendon 626 (as shown), which may comprise all or a portion of the length of the intermediate member 620. In other embodiments, the taper extends only partially along the length of the tendon 626. FIG. 7A shows an enlarged portion of the sidewall of the intermediate member 620 that includes a tapered region of a tendon 626. For ease of explanation, the tendon 626 in FIG. 7A is shown as a solid member, without the branches 626a-626d of FIG. 6. Like FIG. 6, FIG. 7A shows the portion of the intermediate member 620 in the as-manufactured state, i.e., the state directly after removing material from a wall of a tube to make the various features of the intermediate member 620. This as-manufactured state may also represent the tendons 626 in a neutral configuration in which the steerable region 602 is not bent. For those embodiments including locking tendons, the neutral configuration is one in which the locking tendons are in an unlocked state and the tube assembly is flexible, or in which the locking tendons are in a locked state and the tube assembly is rigid, depending on the design of the locking tendons.

The tapered region has a first tangential width W1 at its distal end and a second tangential width W2 at its proximal end where the first width W1 is less than the second width W2. The tapered region has a length TL measured along the longitudinal axis of the intermediate member 620. As previously mentioned, the length of the tapered region may comprise all or a portion of the length of the corresponding tendon 626 and/or all or a portion of the length of the corresponding intermediate member 620, which may comprise all or a portion of the corresponding tube assembly length. It will be appreciated that the greater the width of the tendon 626, the lower the strain/stretch on the tendon 626, which means a more beneficial ratio between input and output.

Along the tapered region, the tendon 626 has a first longitudinal side 656a separated from a first tendon-adjacent portion 627a by a first slit 658a, and a second longitudinal side 656b separated from a second tendon-adjacent portion 627b by a second slit 658b. Both the first and second longitudinal sides 656a, 656b are straight such that the width of the tendon 626 is continuously tapering along the tapered region. The longitudinal sides 657a, 657b of the first and second tendon-adjacent portions 627a, 627b, respectively, may also be straight, and each of the longitudinal sides 657a, 657b may run parallel to the longitudinal side 656a, 656b on the other side of their respective slits 658a, 658b, at least along the tapered region. As a result, each of the first and second slits 658a, 658b can have a substantially constant slit width SW, at least along the tapered region. The slit width SW can be measured across the respective slit in a direction perpendicular to the longitudinal sides defining the respective slit, as shown in FIG. 7A. When the first and second slits 658a, 658b are made by a laser beam, as disclosed herein, the slit width SW is determined by the diameter of the laser beam. In some embodiments, at least in the as-manufactured state, the slit width SW of each slit 658 a, 658 b can be from about 5 μm to about 50 μm or from about 0.015 mm and about 0.04 mm.

Each of the first and second slits 658a, 658b are angled relative to the longitudinal direction L of the intermediate member 630, in some cases at the same but opposite angles. The tapering angle θ may be measured between the respective longitudinal side 657a or 657b and a line parallel to the longitudinal direction L of the intermediate member 620. In some embodiments, the tapering angle 0 is from about 0 degrees to about 5 degrees, or from about 0 degrees to about 2 degrees.

When the intermediate member 620 is in the as-manufactured state, as shown in FIG. 7A, each of the first and second slits 658a, 658b may have a corresponding slit length SL. The slit length SL is measured across the respective slit 658a, 658b from the respective longitudinal side 656a, 656b of the tendon 626 to the longitudinal side 657a, 657b of the respective opposing tendon-adjacent portion 627a, 627b along a line parallel to the longitudinal direction L of the intermediate member 620. The length TL of the tapered region is greater than the individual slit lengths SL of first and second slits 658a, 658b. Moreover, the slit length SL in the as-manufactured state of the intermediate member 620 can be no greater than the functional distance. In some embodiments, the slit length SL is from about 2 mm to about 10 mm, or no more than about 10 mm.

As outlined in FIG. 7B, the relationship between the tapering angle θ, the slit length SL, and the slit width SW can be described using trigonometric ratios for the right triangle formed by the tapering angle θ, the slit length SL, and the slit width SW. The equation becomes: sin(θ)=SW/SL. In some embodiments, the tapering angle θ may be selected based on a desired slit width SW and a desired functional distance. For example, a desired functional distance for a tendon 626 may be from about 2 mm to about 10 mm, and a desired slit width SW may be about 5 μm to about 50 μm. Depending on the functional distance and slit width SW, the tapering angle θ may be about 0.029 degrees (slit width SW=5 μm, functional distance=10 mm) to about 1.433 degrees (slit width SW=50 μm, functional distance=2 mm).

A tendon is configured to perform one or more functions in the instrument, as explained above, including but not limited to deflecting a steerable region of the instrument (a steering tendon) or locking a flexible region of the tube assembly (such as the intermediate portion of the tube assembly) in a certain orientation (a locking tendon). To be able to do so, the tendon 626 is configured to be displaced in the longitudinal direction of the instrument, either away from or towards the first and second tendon-adjacent portions 627a, 627b. When moving the tendon 626 in the direction of the taper the slit width SW and the slit length SL will become smaller. When moving the tendon 626 in the direction opposite that of the taper the slit width SW and slit length SL will become larger.

FIGS. 7C and 7D, for example, show consecutive views of the position of the tendon 626 relative to the first and second tendon-adjacent portions 627a, 627b when the tendon 626 is displaced in the longitudinal direction towards the first and second tendon-adjacent portions 627a, 627b (e.g., the direction of the taper). In the example of FIGS. 7C and 7D, the direction of the taper is the distal direction but, as previously discussed, in some embodiments the taper may be in the proximal direction. Regardless, when the tendon 626 moves axially in the direction of the taper, the slit length SL shortens. Theoretically, the tendon 626 can be moved axially until the tendon 626 contacts the first and second tendon-adjacent portions 627a, 627b and the slit length L becomes 0 μm, at which point additional longitudinal movement of the tendon 626 is impeded. In practice, however, the intermediate member 620 and/or tube assembly may be configured such that the slit length SL always remains greater than or equal to 0 μm when the tendon 626 is displaced in the longitudinal direction towards the first and second tendon-adjacent portions 627a, 627b (i.e., in the direction of the taper). In such embodiments, the intermediate member 620 and/or the associated tube assembly may be configured such that the functional distance of the tendon 626 in the direction of the taper is no greater than the slit length SL.

In some embodiments, the proximal end 660 of the tapered region is continuous with another (not shown) portion of the tendon 626 (e.g., cut from the same tube), which may have a substantially constant width. Such an example is shown in FIGS. 7C and 7D. In some embodiments, the proximal end 660 of the tapered region coincides with the proximal end of the tendon 626, and the proximal end 660 and/or a proximal region of the tapered region may be coupled to an input device (for controlling steering and/or locking/unlocking manually and/or robotically), whether directly or indirectly via another tube positioned outside or inside the tube from which the tendon 626 is made. Example of such coupling techniques can be found in PCT Publication No. WO 2017213491, filed May 31, 2017, which is incorporated by reference herein in its entirety.

Likewise, the distal end 662 (see FIG. 7A) of the tapered region may be continuous with another (not shown) portion of the tendon 626 (e.g., cut from the same tube) which may have a substantially constant width. In some embodiments, the distal end 662 of the tapered region coincides with the distal end of the tendon 626, which may be coupled to a corresponding flexible region 628 (if a steering tendon, as shown) or may terminate along the intermediate portion 620c of the intermediate member 620 (if a locking tendon, not shown).

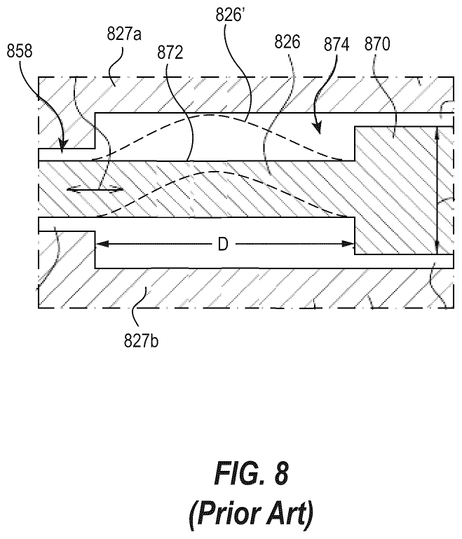

Examples of the present technology provide several technical advantages through the implementation of tapered tendon configurations. In some implementations, the tapered geometry maintains structural integrity by avoiding abrupt cross-sectional transitions that could otherwise lead to stress concentrations and buckling failure modes, particularly in regions where material thickness is reduced. FIG. 8, for example, shows an example tendon 826 having a step-wise change in width, resulting in a first portion 870 having a first width and a second portion 872 having a second width. In order for the tendon 826 to have the space to move longitudinally by the distance D required for steering and/or locking/unlocking, an additional gap 874 needs to be created in the tubular sidewall of the intermediate member, on either side of the tendon 826 between the tendon 826 and the tendon-adjacent portions 827a, 827b. This gap 874 necessarily coincides with the narrower second portion 872 of the tendon 826, thus providing no reinforcement for buckling forces. An example buckled tendon 826′ is shown in FIG. 8. By greatly reducing and/or eliminating buckling, the gradual transition of the tendons of the present technology enhances the durability and operational lifespan of the tendons.

The example shown in FIG. 8 is also not ideal from a manufacturing perspective, especially when the tube is cut with a laser beam having a fixed width. In FIG. 8, the width of the slit 858 represents the width of the laser beam; thus, to make the space necessary to allow practical longitudinal movement of the tendon 826, entire islands of the tubular sidewall must be cut and removed. The gradually tapered tendons of the present technology simplify the manufacturing process by reducing the number of isolated material segments that must be removed following laser processing, while simultaneously decreasing the complexity and length of the laser cutting path. This streamlined fabrication approach can result in reduced processing time and material waste compared to conventional straight-cut tendon designs, improving manufacturing efficiency and cost-effectiveness. Moreover, the taper allows for a gradual transition between wider and narrower sections without requiring excessive room, making the design more compact.

Finally, the tapered design of the present technology offers substantial customization capabilities, enabling optimization of mechanical properties across different sections of the tendon to address specific performance requirements such as payload handling characteristics, fatigue resistance, and accommodation of spatial constraints while preserving smooth navigation through device components (such as spacers) and providing localized stiffness where required for effective steering and/or locking/unlocking control.

### EXAMPLES

The subject technology is illustrated, for example, according to various aspects described below, including with reference to FIGS. 1-8. Various examples of aspects of the subject technology are described as numbered examples (1, 2, 3, etc.) for convenience. These are provided as examples and do not limit the subject technology.

Example 1: An instrument, comprising: an elongate member having a first end portion, a second end portion, and a longitudinal axis extending therebetween, the elongate member comprising a tubular sidewall having: a tendon extending longitudinally along the sidewall and having a width measured between first and second sides of the tendon, the tendon manufactured from the sidewall by forming a first slit in the sidewall at the first side of the tendon and a second slit in the sidewall at the second side of the tendon, wherein the tendon includes a tapered region along which the width of the tendon continuously decreases, a first portion circumferentially adjacent the first side and separated from the tendon by the first slit, a second portion circumferentially adjacent the second side and separated from the tendon by the second slit, wherein each of the first and second slits have a slit length, the slit length being the longest distance measured across the respective slit along a line substantially parallel to the longitudinal axis of the elongate member, and wherein the tapered region has a tapered region length greater than the slit length of the elongate member in an as-manufactured state.

Example 2: The instrument of Example 1, wherein the width of the tendon continuously decreases in a distal direction along the tapered region.

Example 3: The instrument of Example 1 or Example 2, wherein the width of the tendon continuously decreases in a proximal direction along the tapered region.

Example 4: The instrument of any one of Examples 1 to 3, wherein the elongate member has a flexible region at the first end portion, the flexible region formed by a plurality of cuts in the tubular sidewall.

Example 5: The instrument of Example 4, wherein one end of the tendon is coupled to the flexible region such that axial movement of the tendon causes deflection at the first end portion of the elongate member.

Example 6: The instrument of Example 5, wherein the other end of the tendon is configured to be coupled to a steering interface to control axial movement of the tendon.

Example 7: The instrument of any one of Examples 1 to 6, wherein axial movement of the tendon in a first direction causes all or a portion of a length of the elongate member to transform from a flexible configuration to a rigid configuration.

Example 8: The instrument of Example 7, wherein axial movement of the tendon in a second direction opposite the first direction causes the all or a portion of the length of the elongate member to transform from a rigid configuration to a flexible configuration.

Example 9: An instrument, comprising: an elongate member having a first end portion, a second end portion, and a longitudinal axis extending therebetween, the elongate member comprising a tubular sidewall having: a tendon extending longitudinally along the sidewall and having a width measured between first and second sides of the tendon, the tendon manufactured from the sidewall by forming a first slit in the sidewall at the first side of the tendon and a second slit in the sidewall at the second side of the tendon, wherein the tendon includes a tapered region along which the width of the tendon continuously decreases, a first portion circumferentially adjacent the first side and separated from the tendon by the first slit, a second portion circumferentially adjacent the second side and separated from the tendon by the second slit, wherein each of the first and second slits have a slit length, the slit length defined by the longest distance measured across the respective slit along a line substantially parallel to the longitudinal axis of the elongate member, and wherein the tapered region has a tapering angle measured between the line and an edge of the first portion the defines the first slit, the tapering angle being about 0.01 degrees to about 2 degrees.

Example 10: The instrument of Example 9, wherein the width of the tendon continuously decreases in a distal direction along the tapered region.

Example 11: The instrument of Example 9 or Example 10, wherein the width of the tendon continuously decreases in a proximal direction along the tapered region.

Example 12: The instrument of any one of Examples 9 to 11, wherein the elongate member has a flexible region at the first end portion, the flexible region formed by a plurality of cuts in the tubular sidewall.

Example 13: The instrument of Example 12, wherein one end of the tendon is coupled to the flexible region such that axial movement of the tendon causes deflection at the first end portion of the elongate member.

Example 14: The instrument of Example 13, wherein the other end of the tendon is configured to be coupled to a steering interface to control axial movement of the tendon.

Example 15: The instrument of any one of Examples 9 to 14, wherein axial movement of the tendon in a first direction causes all or a portion of a length of the elongate member to transform from a flexible configuration to a rigid configuration.

Example 16: The instrument of Example 15, wherein axial movement of the tendon in a second direction opposite the first direction causes the all or a portion of the length of the elongate member to transform from a rigid configuration to a flexible configuration.

Example 17: An instrument, comprising: an elongate member having a first end portion, a second end portion, and a longitudinal axis extending therebetween, the elongate member comprising a tubular sidewall having: a tendon extending longitudinally along the sidewall and having a width measured between first and second sides of the tendon, the tendon manufactured from the tube by forming a first slit in the sidewall at the first side of the tendon and a second slit in the sidewall at the second side of the tendon, wherein the tendon includes a tapered region along which the width of the tendon continuously decreases, a first portion circumferentially adjacent the first side and separated from the tendon by the first slit, a second portion circumferentially adjacent the second side and separated from the tendon by the second slit, wherein each of the first and second slits have a slit length, the slit length defined by the longest distance measured across the respective slit along a line substantially parallel to the longitudinal axis of the elongate member, and wherein a functional distance of the tendon is less than the slit length of the elongate member in a neutral position, the functional distance comprising an axial displacement required of the tendon to perform a predetermined function as intended during operation of a tube assembly incorporating the elongate member.

Example 18: The instrument of Example 17, wherein the axial displacement is in the direction along which the width of the tendon tapers.

Example 19: The instrument of Example 17 or Example 18, wherein the width of the tendon continuously decreases in a distal direction along the tapered region.

Example 20: The instrument of any one of Examples 17 to 19, wherein the width of the tendon continuously decreases in a proximal direction along the tapered region.

Example 21: The instrument of any one of Examples 17 to 20, wherein the elongate member has a flexible region at the first end portion, the flexible region formed by a plurality of cuts in the tubular sidewall.

Example 22: The instrument of Example 21, wherein one end of the tendon is coupled to the flexible region such that axial movement of the tendon causes deflection at the first end portion of the elongate member.

Example 23: The instrument of Example 22, wherein the functional distance comprises a predetermined axial displacement of the tendon, measured from a position of the tendon when the elongate member is in an as-manufactured state, that is required to cause an intended maximum deflection of the flexible region.

Example 24: The instrument of Example 22, wherein the other end of the tendon is configured to be coupled to a steering interface to control axial movement of the tendon.

Example 25: The instrument of any one of Examples 17 to 24, wherein axial movement of the tendon in a first direction causes all or a portion of a length of the elongate member to transform from a flexible configuration to a rigid configuration.

Example 26: The instrument of Example 25, wherein axial movement of the tendon in a second direction opposite the first direction causes the all or a portion of the length of the elongate member to transform from a rigid configuration to a flexible configuration.

Example 27: The instrument of any Example 26, wherein the functional distance comprises a predetermined axial displacement of the tendon that is required to transform the all or a portion of the length from a rigid configuration to a flexible configuration, or vice versa.

Example 28: An instrument, comprising: a tube with an axis oriented in a longitudinal direction, the tube including a tendon configured to perform a predetermined function and manufactured from the tube by a material removal technique, the tube having a transition zone in which the tendon has a tapered form, the transition zone having a transition zone length, wherein in the transition zone the tendon is separated from a tendon-adjacent portion by a first slit and from a second tendon-adjacent portion by a second slit, and wherein: the second slit is straight and oriented under a tapering angle, θ, θ>0 degrees, relative to the longitudinal direction, the first slit is straight and oriented under a same tapering angle but with opposite sign, relative to the longitudinal direction, the value of sin θ equals a width of the second slit divided by a length of the second slit, wherein the width of the second slit is measured in a manufactured state of the tube and the length of the second slit is measured in the manufactured state of the tube and is less than the transition zone length, and the medical instrument being configured such that the tendon can move in the longitudinal direction towards the first tendon-adjacent portion and the second tendon-adjacent portion along a predetermined longitudinal distance necessary to perform the predetermined function, the predetermined longitudinal distance being smaller than the slot length.

Example 29: The instrument of Example 28, wherein the medical instrument has a distal end and a proximal end, the distal end having at least one deflectable zone and the invasive instrument being configured to deflect the at least one deflectable zone by a movement of the tendon in the longitudinal direction.

Example 30: The instrument of Example 28, wherein the tendon has a lock-unlock function.

Example 31: The instrument of any one of Examples 28 to 30, wherein 0 degrees<θ<5 degrees.

Example 32: The instrument of any one of Examples 28 to 31, wherein 0 degrees<θ<2 degrees.

Example 33: The instrument of any one of Examples 28 to 32, wherein the width of the second slit is from about 5 μm to about 50 μm.

Example 34: The instrument of any one of Examples 28 to 33, wherein the predetermined longitudinal distance necessary to perform the predetermined function is about 2 mm to about 10 mm.

Example 35: The instrument of any one of Examples 28 to 34, wherein the length of the second slit in the manufactured state of the tube is no more than 10 mm.

Example 36: The instrument of any one of Examples 28 to 35, wherein the tube has a tube wall with a wall thickness in a range of 0.03-2.0 mm, 0.03-1.0 mm, 0.05-0.5 mm, or 0.08-0.4 mm.

Example 37: The instrument of any one of Examples 28 to 36, wherein the tube has a tube diameter in a range of 0.5-20 mm, 0.5-10 mm, or 0.5-6 mm.

Example 38: The instrument of any one of Examples 28 to 37, wherein the tube is a first tube and the medical instrument further comprises one or more second tubes coaxially arranged with the first tube.

Example 39: The instrument of Example 38, wherein adjacent coaxial tubes have a radial play in a range of 0.01-0.3 mm.

Example 40: A method of manufacturing an instrument, comprising: providing at least one tube with an axis oriented in a longitudinal direction, applying a material removal technique to make a tendon in the tube, the tendon configured to perform a predetermined function, such that the tube has a transition zone in which the tendon has a tapered form, the transition zone having a transition zone length, wherein in the transition zone the tendon is separated from a first tendon-adjacent portion by a first slit and from a second tendon-adjacent portion by a second slit, the second slit being straight and oriented under a tapering angle, θ, θ>0 degrees, relative to the longitudinal direction, the first slit being straight and oriented under a same tapering angle, −θ, but with opposite sign, relative to the longitudinal direction, wherein a value of sin θ equals a width of the second slit divided by a length of the second slit, wherein the transition zone length is larger than the slot length, designing the medical instrument such that the tendon can move in the longitudinal direction towards the first tendon-adjacent portion and the second tendon-adjacent portion along a predetermined longitudinal distance necessary to perform the predetermined function, the predetermined longitudinal distance being smaller than the length of the second slit.

Example 41: The method of Example 40, wherein the material removal technique is at least one of laser cutting, photochemical etching, deep pressing, or a chipping technique, the chipping technique being one of drilling, milling or high-pressure water jet cutting.

Example 42: The method of Example 40 or Example 41, wherein 0 degrees<θ<5 degrees preferably 0 degrees<θ<2 degrees.

Example 43: The method of any one of Examples 40 to 42, wherein the width of the second slit in the manufactured state of the tube is from about 5 μm to about 50 μm.

Example 44: The method of any one of Examples 40 to 43, wherein the predetermined longitudinal distance necessary to perform the predetermined function is from about 2 mm to about 10 mm.

Example 45: The method of any one of Examples 40 to 44, wherein the length of the second slit in the manufactured state of the tube is no more than 10 mm.

### CONCLUSION

Other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-8.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An instrument, comprising:
an elongate member having a first end portion, a second end portion, and a longitudinal axis extending therebetween, the elongate member comprising a tubular sidewall having:
a tendon extending longitudinally along the sidewall and having a width measured between first and second sides of the tendon, the tendon manufactured from the sidewall by forming a first slit in the sidewall at the first side of the tendon and a second slit in the sidewall at the second side of the tendon, wherein the tendon includes a tapered region along which the width of the tendon continuously decreases,
a first portion circumferentially adjacent the first side and separated from the tendon by the first slit,
a second portion circumferentially adjacent the second side and separated from the tendon by the second slit,
wherein each of the first and second slits have a slit length, the slit length being the longest distance measured across the respective slit along a line substantially parallel to the longitudinal axis of the elongate member, and
wherein the tapered region has a tapered region length greater than the slit length of the elongate member in an as-manufactured state.

2. The instrument of claim 1, wherein the width of the tendon continuously decreases in a distal direction along the tapered region.

3. The instrument of claim 1, wherein the width of the tendon continuously decreases in a proximal direction along the tapered region.

4. The instrument of claim 1, wherein the elongate member has a flexible region at the first end portion, the flexible region formed by a plurality of cuts in the tubular sidewall.

5. The instrument of claim 4, wherein one end of the tendon is coupled to the flexible region such that axial movement of the tendon causes deflection at the first end portion of the elongate member.

6. The instrument of claim 5, wherein the other end of the tendon is configured to be coupled to a steering interface to control axial movement of the tendon.

7. The instrument of claim 1, wherein axial movement of the tendon in a first direction causes all or a portion of a length of the elongate member to transform from a flexible configuration to a rigid configuration.

8. The instrument of claim 7, wherein axial movement of the tendon in a second direction opposite the first direction causes the all or a portion of the length of the elongate member to transform from a rigid configuration to a flexible configuration.

9. An instrument, comprising:
an elongate member having a first end portion, a second end portion, and a longitudinal axis extending therebetween, the elongate member comprising a tubular sidewall having:
a tendon extending longitudinally along the sidewall and having a width measured between first and second sides of the tendon, the tendon manufactured from the sidewall by forming a first slit in the sidewall at the first side of the tendon and a second slit in the sidewall at the second side of the tendon, wherein the tendon includes a tapered region along which the width of the tendon continuously decreases,
a first portion circumferentially adjacent the first side and separated from the tendon by the first slit,
a second portion circumferentially adjacent the second side and separated from the tendon by the second slit,
wherein each of the first and second slits have a slit length, the slit length defined by the longest distance measured across the respective slit along a line substantially parallel to the longitudinal axis of the elongate member, and
wherein the tapered region has a tapering angle measured between the line and an edge of the first portion the defines the first slit, the tapering angle being about 0.01 degrees to about 2 degrees.

10. The instrument of claim 9, wherein the width of the tendon continuously decreases in a distal direction along the tapered region.

11. The instrument of claim 9, wherein the width of the tendon continuously decreases in a proximal direction along the tapered region.

12. The instrument of claim 9, wherein the elongate member has a flexible region at the first end portion, the flexible region formed by a plurality of cuts in the tubular sidewall.

13. The instrument of claim 12, wherein one end of the tendon is coupled to the flexible region such that axial movement of the tendon causes deflection at the first end portion of the elongate member.

14. The instrument of claim 13, wherein the other end of the tendon is configured to be coupled to a steering interface to control axial movement of the tendon.

15. The instrument of claim 9, wherein axial movement of the tendon in a first direction causes all or a portion of a length of the elongate member to transform from a flexible configuration to a rigid configuration.

16. The instrument of claim 15, wherein axial movement of the tendon in a second direction opposite the first direction causes the all or a portion of the length of the elongate member to transform from a rigid configuration to a flexible configuration.

17. An instrument, comprising:
an elongate member having a first end portion, a second end portion, and a longitudinal axis extending therebetween, the elongate member comprising a tubular sidewall having:
a tendon extending longitudinally along the sidewall and having a width measured between first and second sides of the tendon, the tendon manufactured from the tube by forming a first slit in the sidewall at the first side of the tendon and a second slit in the sidewall at the second side of the tendon, wherein the tendon includes a tapered region along which the width of the tendon continuously decreases,
a first portion circumferentially adjacent the first side and separated from the tendon by the first slit,
a second portion circumferentially adjacent the second side and separated from the tendon by the second slit,
wherein each of the first and second slits have a slit length, the slit length defined by the longest distance measured across the respective slit along a line substantially parallel to the longitudinal axis of the elongate member, and
wherein a functional distance of the tendon is less than the slit length of the elongate member in a neutral position, the functional distance comprising an axial displacement required of the tendon to perform a predetermined function as intended during operation of a tube assembly incorporating the elongate member.

18. The instrument of claim 17, wherein the axial displacement is in the direction along which the width of the tendon tapers.

19. The instrument of claim 17, wherein the width of the tendon continuously decreases in a distal direction along the tapered region.

20. The instrument of claim 17, wherein the width of the tendon continuously decreases in a proximal direction along the tapered region.
